(19) United States
(12) Patent Application Publication
(10) Pub. No.: US 2023/0253094 A1
(43) Pub. Date: Aug. 10, 2023

(54) PREEMPTIVE IMAGE DATA CACHING BASED ON GEOLOCATION

(71) Applicant: Change Healthcare Holdings, LLC, Nashville, TN (US)

(72) Inventors: Vladan Petrovic, Burnaby (CA); Valentin Pirvulescu, New Westminster (CA)

(21) Appl. No.: 17/665,642

(22) Filed: Feb. 7, 2022

(51) Int. Cl. G16H 30/20; G06T 1/60; H04W 4/02; G06F 16/51; G06F 16/53; G06F 16/587

(52) U.S. Cl. CPC G16H 30/20; G06T 1/60; H04W 4/023; G06F 16/51; G06F 16/53; G06F 16/587

(57) ABSTRACT

A system for caching images for remote viewing includes a memory device having instructions stored thereon that, when executed by one or more processors, cause the one or more processors to perform operations including receiving first location data from a user device associated with a user, determining whether the user is within a threshold distance of a remote image viewing device based on the first location data, identifying a local caching device associated with the remote image viewing device responsive to a determination that the user is within the threshold distance of the remote image viewing device, and transmitting image data associated with the user to the local caching device.

### BACKGROUND

The present disclosure relates generally to enterprise data caching, and more specifically to caching image data for local viewing. Large and often remote enterprise imaging data centers can store enormous amounts of image data for viewing on local devices (e.g., desktop computers, laptop computers, mobile phones, tablets, etc.). In a healthcare setting, for example, medical images used by image-centric physicians (e.g., radiologists, surgeons, orthopedists, cardiologists, oncologists, etc.) for diagnosing ailments and for making clinical decisions may be stored in an off-site data center (e.g., a remote server) and later retrieved by the physicians for viewing. In some cases, image data must be retrieved from a data center in a matter of seconds; however, many current solutions lack the ability to quickly retrieve a large number of images due to, for example, network latencies and bandwidth constructions when transmitting image data from a remote data center to an on-premise data center or local image viewing device. Additionally, many current solutions cache image data real-time, such as when a user is viewing images on a local device, which not only utilizes valuable computing resources but can also lead to decreased performance.

### SUMMARY

One implementation of the present disclosure is a system for caching images for remote viewing. The system includes a memory device having instructions stored thereon that, when executed by one or more processors, cause the one or more processors to perform operations including receiving first location data from a user device associated with a user, determining whether the user is within a threshold distance of a remote image viewing device based on the first location data, identifying a local caching device associated with the remote image viewing device responsive to a determination that the user is within the threshold distance of the remote image viewing device, and transmitting image data associated with the user to the local caching device.

In some embodiments, the operations further include determining an identifier for a particular cache of the local caching device and transmitting the identifier to the user device, the image data being transmitted to the particular cache based on the identifier.

In some embodiments, the identifier is a web address or a link for the particular cache of the local caching device.

In some embodiments, the operations further include receiving, from the user device, metadata associated with the user and identifying the image data associated with the user based on the metadata.

In some embodiments, identifying the image data further includes querying an image database based on the metadata associated with the user and retrieving the image data from the image database.

In some embodiments, the first location data is received at a first time and the operations further include receiving second location data from the user device at a second time that is after the first time and determining whether the user is traveling to the remote image viewing device based on the first location data and the second location data, the location caching device being further identified responsive to a determination that the user is traveling to the remote image viewing device.

In some embodiments, the operations further include transmitting an acknowledgement to the user device responsive to identifying the local caching device.

In some embodiments, the local caching device is a computing device located at a healthcare facility.

In some embodiments, the image data is medical image data associated with one or more patients of the user.

Another implementation of the present disclosure is a method of caching image data. The method includes receiving, by a processor, location data from a mobile device associated with a user, determining, by the processor, whether the user is within a threshold distance of a remote image viewing device, identifying, by the processor, a caching service associated with the remote image viewing device responsive to a determination that the user is within the threshold distance of the remote image viewing device, and transmitting, by the processor, the image data to the caching service.

In some embodiments, the method further includes determining, by the processor, an identifier for a particular cache of the caching service and transmitting, by the processor, the identifier to the user device, the image data being transmitted to the particular cache based on the identifier.

In some embodiments, the identifier is a web address or a link for the particular cache.

In some embodiments, the method further includes receiving, by the processor, metadata associated with the user from the user device and identifying, by the processor, the image data associated with the user based on the metadata.

In some embodiments, identifying the image data further includes querying, by the processor, an image database based on the metadata associated with the user and retrieving, by the processor, the image data from the image database.

In some embodiments, the first location data is received at a first time, the method further including receiving, by the processor, second location data from the user device at a second time that is after the first time and determining, by the processor, whether the user is traveling to the remote image viewing device based on the first location data and the second location data, the caching service being further identified responsive to a determination that the user is traveling to the remote image viewing device.

In some embodiments, including transmitting an acknowledgement to the user device responsive to identifying the caching service.

In some embodiments, the caching service is hosted by a computing device located at a healthcare facility.

In some embodiments, the image data is medical image data associated with one or more patients of the user.

Yet another implementation of the present disclosure is a non-transitory computer readable media having instructions stored thereon that, when executed by one or more processors, cause the one or more process to perform operations including continuously receiving location data from a user device associated with a user, determining, based on the continuously received location data, whether the user is i) within a threshold distance of a remote image viewing device, or ii) traveling to the remote image viewing device, identifying a local image caching device associated with the remote image viewing device responsive to a determination that the user is i) within the threshold distance of the remote image viewing device, or ii) traveling to the remote image viewing device, identifying image data associated with the user, and transmitting the image data to the local image caching device.

In some embodiments, the location data is received at a regular time interval.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is an example architecture of an image caching system without preemptive image caching, according to some embodiments.

FIG. 2 is an example architecture of an image caching system with preemptive image caching, according to some embodiments.

FIG. 3 is a diagram of a system for preemptive image caching based on a user's location, according to some embodiments.

FIG. 4 is a diagram of a picture archiving and communication system (PACS) for preemptively caching image data, according to some embodiments.

FIG. 5 is a process for trigging image caching based on a user's location, according to some embodiments.

FIG. 6 is a diagram of illustrating the triggering of image caching based on a user's location, according to some embodiments.

FIG. 7 is a process for identifying a caching location based on a user's location, according to some embodiments.

FIG. 8 is a process for caching image data, according to some embodiments.

### DETAILED DESCRIPTION

Referring generally to the FIGURES, a system and methods preemptively caching image data for local viewing based on a user's location are shown, according to various embodiments. Advantageously, the system and method described herein may alleviate many of the issues discussed throughout this disclosure by triggering the caching of image data on a local device (e.g., a local computing device such as a server) when a user (e.g., a physician) is at, near, or traveling to an available image viewing device. In particular, a user's location may be evaluated to determine whether the user is within a threshold distance of a known image viewing device or whether the user is traveling to a known image viewing device. If it is determined that the user is within the threshold distance or that the user is traveling to an image viewing device, image data caching may be triggered causing image data to be cached on a local caching device (e.g., an on-premise server), thereby reducing the latency and performance issues associated with real-time image caching.

In contrast, other systems may cache image based on trivial heuristics or statistic policies that, quite often, have the potential of being incorrect or inefficient. For example, in a healthcare setting, image data may be cached locally during out-of-peak hours (e.g., overnight) or at a predetermined time (e.g., before working hours, before a scheduled shift, etc.). These types of systems and methods can require a great deal of computing resources for retaining image data in a local cache and can stifle network traffic during peak hours, especially in adaptive environments such hospitals. Additionally, due to the distance between a local image viewer and a remote image data center, interactive performance may be greatly degraded. For example, scrolling, zooming, panning, or otherwise manipulating viewed images on a local image viewer may cause distortions such as skipping or lagging.

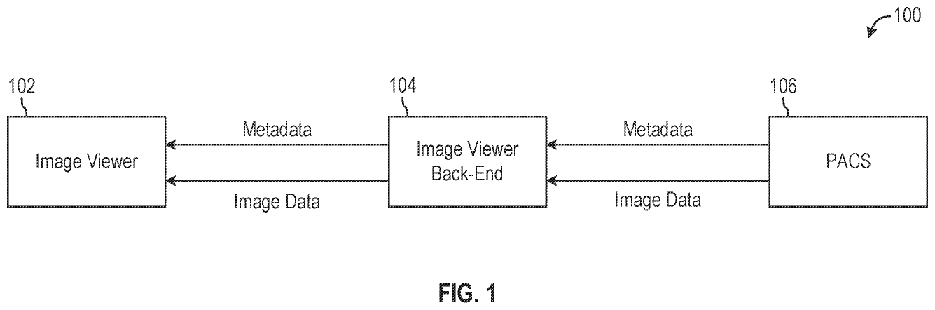

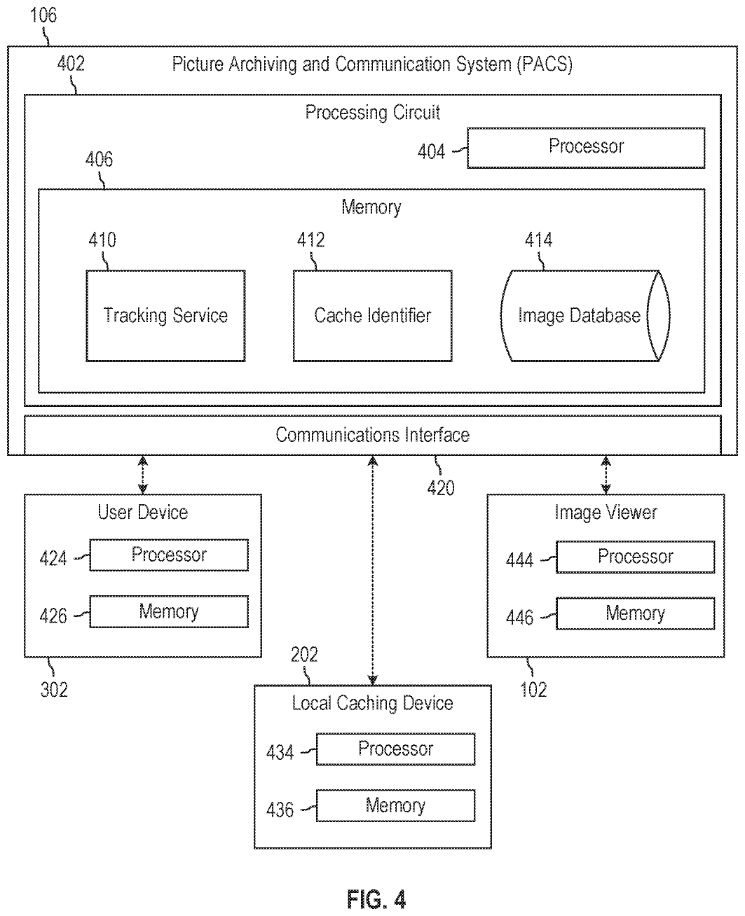

Turning first to FIG. 1, an example architecture of an image caching system 100 without preemptive image caching is shown, according to some embodiments. Specifically, system 100 may be an example of a traditional image caching system, such as one utilized in the healthcare industry by physicians and/or other medical professionals, for viewing image data (e.g., related to a patient) on an image viewing device, shown as image viewer 102. Image viewer 102 may be any device capable of receiving image data and reproducing an image from the image data. Accordingly, image viewer 102 may include a user interface which further includes a display for displaying images and/or a user input device such as a keyboard, a mouse, a touchscreen, etc. Image viewer 102 may also include at least one processor and memory device for performing operations such as receiving user inputs, receiving (i.e., downloading) image data, converting image data into images to be displayed on a user interface, etc. For example, image viewer 102 may be a desktop computer, a laptop computer, a mobile phone (e.g., a smartphone), a tablet, or any other suitable computing device. Additional features of image viewer 102 are described in greater detail below with respect to FIG. 4.

System 100 is also shown to include an image viewer back-end 104, which may include any back-end devices or services utilized by image viewer 102 to receive and display image data. For example, image viewer back-end 104 may include a separate computing device, such as a server, that can transmit and receive data (i.e., communicate) with image viewer 102. In another example, image viewer back-end 104 may be a portion of image viewer 102 itself. In any case, image viewer back-end 104 may generally be positioned locally to image viewer 102, such as within the same facility, building, or complex, thereby acting as an intermediary between image viewer 102 and a remote picture archiving and communication system (PACS) 106. For example, image viewer back-end 104 may be an on-site server at a hospital or that serves multiple hospitals, and that can be accessed by one or more image viewers (e.g., multiple of image viewer 102). Accordingly, image viewer back-end 104 may also include at least one processor and memory device for performing operations such as receiving and storing image data and communicating with one or both of image viewer 102 and PACS 106.

PACS 106, as described in greater detail below, is a system for storing and processing image data. In some embodiments, PACS 106 is a computing device (e.g., a server, a computer, etc.) that is remote from one or both of image viewer 102 and image viewer back-end 104. For example, PACS 106 may be a server that is hosted off-site from image viewer 102. In some such embodiments, PACS 106 may be a cloud server that is communicably coupled to image viewer 102 and/or image viewer back-end 104 via a network, such as the Internet. Image data may be transmitted to PACS 106 for storage and/or processing from any image-collecting source. In a hospital, for example, image data may be transmitted to PACS 106 from various medical imaging devices such as magnetic resonance imagers (MRIs), ultrasounds, x-ray machines, etc. In some embodiments, image data may be first collected by an intermediary device, such as image viewer back-end 104 or another computing device, before being transmitted to PACS 106. In any case, PACS 106 may receive image data from any number of imaging devices (e.g., medical imaging devices) which may be stored in a database.

In some embodiments, PACS 106 may also receive and store metadata associated with the image data and/or metadata for a user associated with the image data. Metadata can include any additional data relating to the image data, such as information about an image file (e.g., time of capture, size, file type, etc.), as well as patient, study, or series data relating to an image. A study, as described herein, may be a set of information and/or images collected during a medical imaging procedure and related to a particular patient or the procedure itself. A series may be a subset of images with a common attribute. For example, a series may include a plurality of images from a particular region of a patient's body. Accordingly, metadata may include information such as a name and/or identifier for the physician that captured the images, a time and/or date that the image was captured, a patient's name and/or an identifier for the patient (e.g., an ID number), details about the patient (e.g., age, height, weight, etc.), details about the medical imaging device that captured the image (e.g., type, model number, etc.), or any other relevant data associated with the physician, patient, or captured images.

Still referring to FIG. 1, PACS 106 is shown to transmit both metadata and image data to image viewer back-end 104. Likewise, image viewer back-end 104 is shown to transmit metadata and image data to image viewer 102 for viewing. In some embodiments, image viewer back-end 104 caches the metadata and image data prior to transmitting to image viewer 102. For example, image data may be cached based on predetermined caching policies, such as those described above. In such embodiments, image viewer back-end 104 may cache image data based on a predetermined schedule or at a predetermined time. As described above, caching data based solely on static policies can often lead to inefficiencies, such as caching image data a significant amount of time prior to the image data being utilized, thereby causing image viewer back-end 104 to waste resources retaining image data for long periods of time.

In other embodiments, image data may be transmitted from PACS 106 to image viewer back-end 104 and/or from image viewer back-end 104 to image viewer 102 on-demand. In other words, image data may be downloaded and/or streamed from PACS 102 to image viewer back-end 104 and/or image viewer 102 in real-time. As discussed briefly above, the large physical distances between image viewer 102, image viewer back-end 104, and PACS 106 can lead to degraded interactive performance when viewing images. For example, when scrolling, zooming, or panning on a viewed image (e.g., displayed on image viewer 102), network latencies and bandwidth restrictions may cause the viewed images to stutter, freeze, or lag. However, it is worth noting that metadata, which is typically smaller in file size and may not be as readily manipulated as an image, may not necessarily be affected by network latency and bandwidth restrictions as image data.

### Preemptive Data Caching

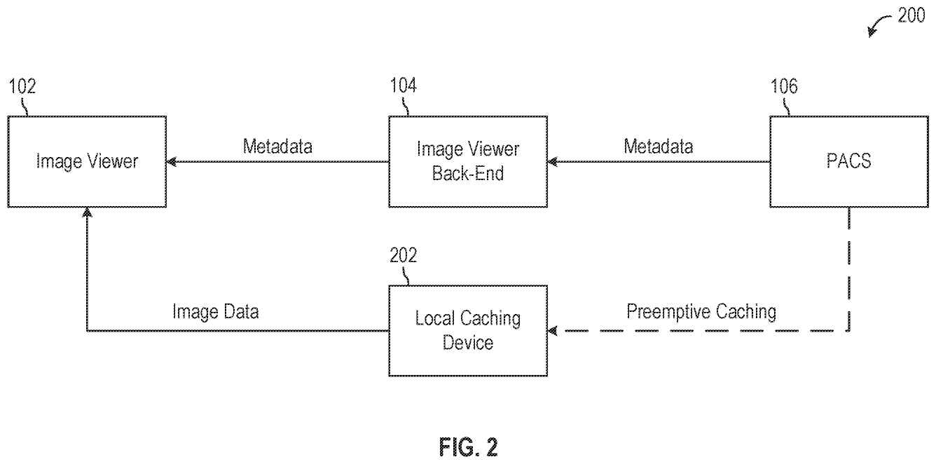

Referring now to FIG. 2, an example architecture of an image caching system 200 with preemptive image caching is shown, according to some embodiments. By preemptively caching image data before a user (e.g., a physician) reaches or interacts with image viewer 102, system 200 may address many of the shortcomings of system 100 discussed above. Specifically, image caching may be triggered (i.e., initiated) based on a user's detected location to ensure that images are readily available for viewing without degraded performance, while also preventing image data from being cached too early which, as discussed above, can waste valuable computational resources.

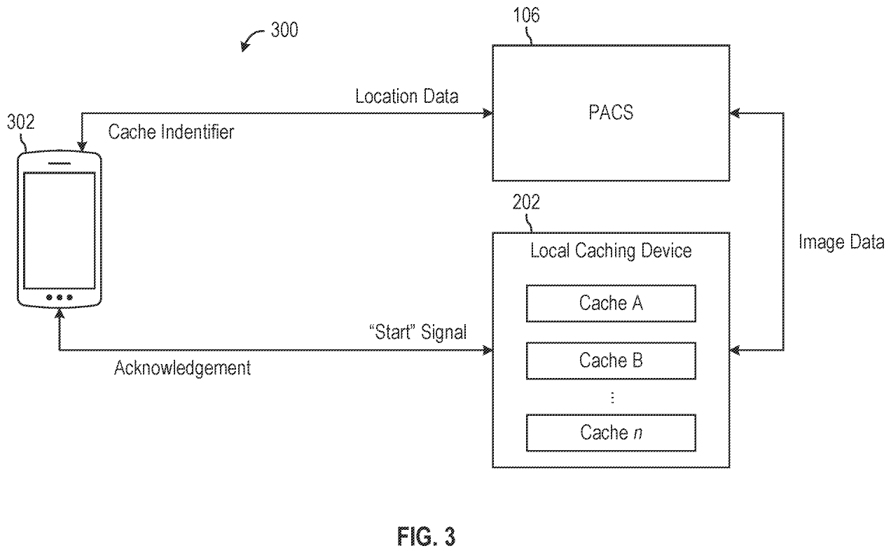

System 200 may include a local caching device 202 configured to preemptively cache image data from PACS 106. Local caching device 202 is, in general, any computing device capable of receiving, storing, and transmitting image data. Accordingly, local caching device 202 can include at least one processor and a memory device for performing operations including, but not limited to, receiving, storing, and transmitting image data. Although shown as a separate component of system 200, in some embodiments, local caching device 202 is a portion of image viewer back-end 104 or image viewer back-end 104 may implement the operations of local caching device 202 described herein. In other embodiments, local caching device 202 is a separate device (e.g., a server) from image viewer back-end 104. However, it will be appreciated that image viewer back-end 104 and local caching device 202 may share implementation of any of the operations described herein. Additional features of local caching device 202 are described in greater detail below with respect to FIGS. 3 and 4.

In some embodiments, local caching device 202 is collocated with one or both of image viewer 102 and image viewer back-end 104. For example, local caching device 202 may be located within the same building (e.g., hospital), facility, campus, or geographical area as image viewer 102. For example, local caching device 202 may be an on-site server or other computing device located in the same hospital building as image viewer 102. In some embodiments, local caching device 202 is considered “local” because it is communicably coupled to image viewer 102 and/or image viewer back-end 104 via any suitable local network (e.g., a local area network (LAN), a wide area network (WAN), a virtual private network (VPN), etc.). For example, local caching device 202 may be server or other computing device communicably coupled to a hospital's internal network (i.e., intranet).

As shown, local caching device 202 may be configured to preemptively cache image data from PACS 106 based on a determination that a user, such as a physician, is physically located at or near image viewer 102. For example, the user may be standing at or near image viewer 102, may be in the same room or area as image viewer 102, or may be in the same building as image viewer 102. In some embodiments, imaging caching is triggered based on a determination that the user is traveling (i.e., moving) to image viewer 102. For example, user location data may be collected over time (e.g., at regular intervals) and used to determine a trajectory or path that the user is traveling. In any case, image data may be cached prior to the user interacting with (e.g., viewing images on) image viewer 102 to prevent the performance issues described above.

Those of ordinary skill in the art will note, however, that it may not be necessary to preemptively cache metadata associated with the image data, as shown in FIG. 2. As described above, metadata may include any type of information relating to an image, including image characteristics and parameters and, in a hospital setting, information relating to the physician(s) and/or patient associated with the image data. Accordingly, metadata is typically much smaller in file size than image data. Thus, the impact on the performance of system 200 due to the downloading or caching of metadata in real-time or on-demand may be negligible. However, in some embodiments, metadata may also be cached preemptively based on a user's location.

Referring now to FIG. 3, a diagram of a system 300 for preemptive image caching based on a user's location is shown, according to some embodiments. In some regards, system 300 illustrates certain aspects of system 200 in greater detail. For example, system 300 is shown to include PACS 106 and local caching device 202, as briefly described above. Additionally, system 300 is shown to include a user device 302. User device 302 may be any computing device that is operable by a user to interact with PACS 106 and/or local caching device 202. Generally, user device 302 may include a user interface, such as a screen for displaying images and text and, in some embodiments, an input device (e.g., a mouse, a keyboard, a keypad, a touchscreen, etc.) for receiving user inputs. For example, user device 302 may be a smartphone associated with a user (e.g., a smartphone carried by a physician in a hospital). Additional details regarding user device 302 are described in greater detail below with respect to FIG. 4.

In some embodiments, a user's location is determined by PACS 106 based on location data transmitted from user device 302 to PACS 106. As described herein, the user's location may refer to a geographical location of user device 302, which is typically carried by a user (e.g., a physician) during use. In such embodiments, location data may be transmitted to PACS 106 as geographical coordinates, although it will be appreciated by those of ordinary skill in the art that any format of location data that describes the user's geographical location may be used. In some embodiments, location data also includes a location time and/or a velocity of the user, which may be determined based on accelerometer data and/or regular or continuous location measurements (e.g., via GPS). Based on the received location data, PACS 106 may be configured to determine whether the user is near (e.g., within a threshold distance of) an image viewing device (e.g., image viewer 102). If it determined that the user is not within a threshold distance from an image viewing device, PACS 106 may also be configured to determine whether the user is moving towards an image viewing device. For example, PACS 106 may compare location data from two different time steps to determine a trajectory of the user.

In some embodiments, rather than receiving location data directly from user device 302, PACS 106 may simply receive an indication from user device 302 that the user is near, or is traveling to, an image viewing device. In such embodiments, user device 302 may determine the user's position and/or trajectory (e.g., based on GPS data) and may compare the user's position and/or trajectory with known locations of image caching devices and/or local caching devices. For example, user device 302 may download and/or reference a list of image caching device and/or local caching device locations to determine whether the user is near, or is traveling to, an image viewing device.

If it is determined that the user is either near an image viewing device or moving towards and image viewing device, PACS 106 and/or user device 302 may be configured to identify a nearest image caching device, such as local caching device 202. For example, local caching device 202 may be identified by comparing a known location of local caching device 202 with a current location of the user. In addition to identifying a nearest image caching device, PACS 106 may be configured to determine a particular cache within the image caching device. As shown, for example, local caching device 202 may have a plurality of separate caches, or memory partitions, for caching sets of data. In this manner, related data sets (e.g., a set of images relating to a particular patient) may be cached in a common location.

Once a caching device and/or particular cache has been identified, PACS 106 may be configured to transmit a cache identifier to user device 302. A cache identifier may be a web address (e.g., uniform resource locator (URL)) or another similar type of identifier, for example. In some cases, such as when it is determined that the user is not near or traveling to an image viewing device, or when an available local caching device cannot be found, PACS 106 may be configured to transmit an acknowledgement (“ACK”) signal back to user device 302. The acknowledgement may indicate that PACS 106 has received usable location data from user device 302 but that a local caching device has not been identified.

Responsive to receiving a cache identifier (e.g., a URL) from PACS 106, user device 302 may be configured to transmit a “start” signal or command to the identified local caching device (e.g., local caching device 202) using the cache identifier, thereby trigging the caching of image data to the identified caching device. Additionally, in some embodiments, user device 302 is configured to transmit metadata to local caching device 202. This metadata may include, for example, an identifier (e.g., a name, an ID number, etc.) for the user associated with user device 302. Local caching device 202 may subsequently being transferring (i.e., downloading) image data from PACS 106. In some embodiments, local caching device 202 transmits a query or command to PACS 106 to cause PACS 106 to retrieve and transmit (e.g., to local caching device 202) relevant image data. In some such embodiments, the query may include the identifier for the user associated with the caching request, thereby allowing PACS 106 to retrieve only the image data associated with the user.

Once image caching has been triggered (i.e., once local caching device 202 transmits a query to PACS 106 and/or begins downloading image data), local caching device 202 may be configured to transmit a second acknowledgement signal to user device 302. In some embodiments, local caching device 202 can delay the triggering of image caching and/or may decide not to cache image data for a variety of reason. For example, local caching device 202 may be configured to follow one or more predefined rules that may prevent the triggering of imaging caching. In such embodiments, local caching device 202 may be configured to transmitting a negative acknowledgement signal (“NACK”) to user device 302, indicating that the “start” signal was received image caching was not triggered (i.e., started).

Referring now to FIG. 4, a diagram of PACS 106 is shown in greater detail, according to some embodiments. As described above, PACS 106 may be configured to receive, maintain, and transmit image data and, in some cases, metadata associated with said image data. In some embodiments, PACS 106 is located remotely from one or more image caching devices and/or image viewers. For example, PACS 106 may be located and hosted at a location remote (i.e., off-site) from one or more hospitals or other facilities that each have one or more on-site (i.e., local) caching devices and/or image viewing devices. Accordingly, PACS 106 may be any suitable remote computing device, such as a server, for performing the various operations described herein. However, it will also be appreciated that any of the functionality described herein with respect to PACS 106 may be implemented using one or more local caching devices, image viewers, user devices, or other computing devices. Additionally, it will be appreciated that PACS 106 may implement additional functionality than that described with respect to FIG. 4.

PACS 106 is shown to include a processing circuit 402 that includes a processor 404 and memory 406. Processor 404 can be a general-purpose processor, an application specific integrated circuit (ASIC), one or more field programmable gate arrays (FPGAs), a group of processing components, or other suitable electronic processing components. In some embodiments, processor 404 is configured to execute program code stored on memory 406 to cause PACS 106 to perform one or more operations as described herein. Memory 406 can include one or more devices (e.g., memory units, memory devices, storage devices, etc.) for storing data and/or computer code for completing and/or facilitating the various processes described in the present disclosure.

In some embodiments, memory 406 includes tangible, computer-readable media that stores code or instructions executable by processor 404. Tangible, computer-readable media refers to any media that is capable of providing data that causes PACS 106 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 406 can include random access memory (RAM), read-only memory (ROM), hard drive storage, temporary storage, non-volatile memory, flash memory, optical memory, or any other suitable memory for storing software objects and/or computer instructions. Memory 406 can include database components, object code components, script components, or any other type of information structure for supporting the various activities and information structures described in the present disclosure. Memory 406 can be communicably connected to processor 404, such as via processing circuit 402, and can include computer code for executing (e.g., by processor 404) one or more processes described herein.

While shown as individual components, it will be appreciated that processor 404 and/or memory 406 can be implemented using a variety of different types and quantities of processors and memory. For example, processor 404 may represent a single processing device or multiple processing devices. Similarly, memory 406 may represent a single memory device or multiple memory devices. Additionally, in some embodiments, PACS 106 may be implemented within a single computing device (e.g., one server, one housing, etc.). In other embodiments PACS 106 may be distributed across multiple servers or computers (e.g., that can exist in distributed locations). For example, PACS 106 may include multiple distributed computing devices (e.g., multiple processors and/or memory devices) in communication with each other that collaborate to perform operations. In another example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 406 is shown to include a tracking service 410 configured to determine a user's location. Specifically, tracking service 410 may be configured to receive location data from user device 302 either on-demand or at regular time intervals (e.g., every second, every few seconds, every minute, etc.). Location data from user device 302 may be used to infer a location of a user. For example, user device 302 may be a device carried by a user, such as a smartphone or smartwatch; thus, the location of user device 302 may correspond to the location of a user. In this manner, tracking service 410 may be configured to track a user's location, either in real time or over a period of time. In some embodiments, tracking service 410 receives user location data in the form of geographical coordinates. In other embodiments, tracking service 410 receives user location data as a street address, building number, room or space identifier, etc. For example, tracking service 410 may determine a room number that the user is in rather than a geographical location. It will be appreciated that tracking service 410 may receive and/or represent location data in any suitable format.

In some embodiments, tracking service 410 is configured to determine a location of user device 302 at a current time step without saving location data. In such embodiments, user privacy may be protected by not saving location data for a user over time. In some embodiments, location data is collected for a limited period of time before being purged (i.e., deleted). For example, tracking service 410 may collect location data at a predetermined number of time steps (e.g., 10 time steps, 100 time steps) or over a predetermined amount of time (e.g., over 10 minutes) in order to detect a trajectory or path that the user is traveling. In such embodiments, location data may be deleted after the user's trajectory is determined. In yet other embodiments, tracking service 410 may be configured to detect and store user location data indefinitely or over a greater period of time (e.g., days, weeks, months) to learn habits of the user.

In some embodiments, tracking service 410 is also configured to predict a user's future location based on data received from external systems (not shown). For example, tracking service 410 may receive scheduling information for a hospital that identifies dates and times that one or more users are scheduled to work. Using this information, tracking service 410 may be able to determine when a user (e.g., a physician) will arrive at the hospital (i.e., will be in a particular location). In another example, tracking service 410 may reference surgery schedules or scheduled appointments to determine when the physician will be in particular rooms or areas of the hospital. Additionally, while shown in FIG. 4 as a component of PACS 106, it will be appreciated that, in other embodiments, tracking service 410 may be a component of user device 302 (e.g., implemented via memory 426, as described below).

Memory 406 is also shown to include a cache identifier 412 configured to identify a nearest local caching device to the user and, in some embodiments, a particular cache for caching image data. In some embodiments, cache identifier 412 compares location data (e.g., either real-time or predicted location data) to a list of known caching devices. For example, location data may be used to query a table stored in a database of PACS 106 (not shown) to identify a closest local caching device. In some embodiments, cache identifier 412 is also configured to identify a particular cache of a local caching device that is available for caching image data. For example, cache identifier 412 may determine an available amount of storage space for one or more caches, which can be compared to a known file size for image data that is to be cached in order to identify a particular caching location. In some embodiments, cache identifier 412 may return (e.g., transmit to user device 302) an identifier for an identified caching location. The identifier may be a web address (e.g., URL) or any other suitable format for identifying the particular cache.

Memory 406 is also shown to include an image database 414, which maintains (i.e., stores and retrieves) image data. As described herein, image data may generally refer to medical imaging data collected from any number of medical imaging devices (e.g., MRIs, x-rays, CT scanners, etc.). Image data may include one or more medical images, typically from a single patient, and in some embodiments can include metadata associated with the medical images. Metadata may include, for example, information about the physician that captured the images (e.g., name, identifier, location, etc.), information about the patient associated with the images (e.g., name, identifier, age, gender, etc.), and/or information about the images themselves (e.g., file size, resolution, time and location of capture, an identifier for the capturing medical imaging device, an area of the body related to the images, etc.).

In some embodiments, image data is received by PACS 106 directly from remote medical imaging devices. For example, any number of medical imaging devices may automatically transmit imaging data to PACS 106 via a network connection (e.g., the Internet). In other embodiments, image data is received from an intermediary source, such as a local caching device (e.g., local caching device 202) or a local server after having been uploaded to the local caching device or server from the medical imaging equipment.

Still referring to FIG. 4, PACS 106 is also shown to include a communications interface 420. Communications interface 420 may facilitate communications between PACS 106 and any external components or devices. For example, communications interface 420 can provide means for transmitting data to, or receiving data from, one or more of user device 302, local caching device 202, and image viewer 102, all briefly described above. Accordingly, communications interface 420 can be or can include a wired or wireless communications interface (e.g., jacks, antennas, transmitters, receivers, transceivers, wire terminals, etc.) for conducting data communications. In some embodiments, communications interface 420 may also provide power to various external components.

In various embodiments, communications via communications interface 420 may be direct (e.g., local wired or wireless communications) or via a network (e.g., a WAN, the Internet, a cellular network, etc.). For example, communications interface 420 can include a WiFi transceiver for communicating via a wireless communications network. In another example, communications interface 420 may include cellular or mobile phone communications transceivers. In yet another example, communications interface 420 may include a low-power or short-range wireless transceiver (e.g., Bluetooth®). Thus, it will be appreciated that communications interface 420 may be configured to transmit and receive data by any suitable means and/or on any suitable try of network.

As briefly described above, user device 302 may be any computing device that is operable by a user to interact with PACS 106. For example, user device 302 may be a personal computer (e.g., laptop or desktop), a smartphone, a tablet, a smartwatch, etc. In general, user device 302 may include a user interface (not shown) that displays text and images and, in some cases, receives user inputs. For example, user device 302 may be a smartphone with a touchscreen. In some embodiments, user device 302 includes a positioning system (not shown) or components configured to detect the device's geographical location. In some such embodiments, user device 302 may include a global positioning system (GPS) transceiver configured to communicate with GPS satellites to determine a location. In some embodiments, user device 302 can determine a location using other types of positioning components. For example, user device 302 may determine a location by triangulating received radio signals, such as from a cellular network, a WiFi® network, or the like.

Local caching device 202 may be any computing device configured to receive, store, and/or transmit image data. For example, local caching device 202 may be a server coupled to PACS 106 via a network (e.g., a WAN or VPN) configured to receive and store image data from PACS 106. In some embodiments, image data may be stored temporarily (e.g., for a predetermined amount of time; although, it will be appreciated that local caching device 202 may store image data for any amount of time. In some embodiments, local caching device 202 is also configured to cache metadata, as described above. In some embodiments, local caching device 202 can maintain a database of images and/or metadata. In some such embodiments, local caching device 202 may maintain a plurality of individual caches that can be used to collocate received data. As described herein, local caching device 202 may be “local” to (i.e., located at) a particular building, facility, or geographical area. In some cases, local caching device 202 may be considered “local” by being connected to a limited network, such as a WAN, LAN, or VPN. For example, local caching device 202 may be physically located in a hospital building or hospital campus and/or local caching device 202 may be connected to an individual hospital or hospital group network.

Image viewer 102 may be any computing device configured to process and display image data as graphics. For example, image viewer 102 may be a computer (e.g., a laptop or desktop), a tablet, a smartphone, or the like that includes a display screen (not shown) for displaying images. In some embodiments, image viewer 102 is located in a particular building, space, or room associated with local caching device 202. For example, image viewer 102 may be in a particular room of a hospital served by local caching device 202. In some embodiments, a hospital or other type of facility may include a plurality of image viewers. In some embodiments, rather than or in addition to being located in the same building as local caching device 202, image viewer 102 may simply be communicably coupled to the same network as local caching device 202. For example, image viewer 102 may communicate with local caching device 202 and/or PACS 106 via an internal network (e.g., LAN or VPN).

Each of user device 302, local caching device 202, and image viewer 102 are shown to include a processor and memory. Specifically, user device is shown to include a processor 424 and memory 426; local caching device 202 is shown to include a processor 434 and memory 436; and image viewer 102 is shown to include a processor 444 and memory 446. Like processors 404 described above, each of processors 424, 434, and 444 may be general-purpose processors, ASICS, FPGAs, groups of processing components, or other suitable electronic processing components. In some embodiments, processors 424, 434, and 444 are configured to execute program code stored on a corresponding one of memory 426, 436, and 446 to cause the corresponding device to perform one or more operations as described herein.

Likewise, each of memory 426, 436, and 446 can include one or more devices (e.g., memory units, memory devices, storage devices, etc.) for storing data and/or computer code for completing and/or facilitating the various processes described in the present disclosure. In some embodiments, memory 426, 436, and 446 includes tangible, computer-readable media that stores code or instructions executable by processor 404. Tangible, computer-readable media refers to any media that is capable of providing data that causes a corresponding device to operate in a particular fashion. Memory 426, 436, and 446 can include random access memory (RAM), read-only memory (ROM), hard drive storage, temporary storage, non-volatile memory, flash memory, optical memory, or any other suitable memory for storing software objects and/or computer instructions. Memory 426, 436, and 446 can include database components, object code components, script components, or any other type of information structure for supporting the various activities and information structures described in the present disclosure.

Additionally, while not shown in FIG. 4, each of user device 302, local caching device 202, and image viewer 102 may include a respective communications interface, similar in functionality to communications interface 420. Specifically, each of user device 302, local caching device 202, and image viewer 102 may be configured to communicate with PACS 106 via any suitable wired or wireless network, as described above. For example, user device 302, which may be a smartphone carried by a user, may include a wireless communications interface for communicating via a cellular or other wireless network. As another example, local caching device 202 may be communicably coupled to PACS 106 via a wired network connection (e.g., a LAN). In some embodiments, one or more of user device 302, local caching device 202, and image viewer 102 may communicate with PACS 106 via the Internet, such as via a web portal.

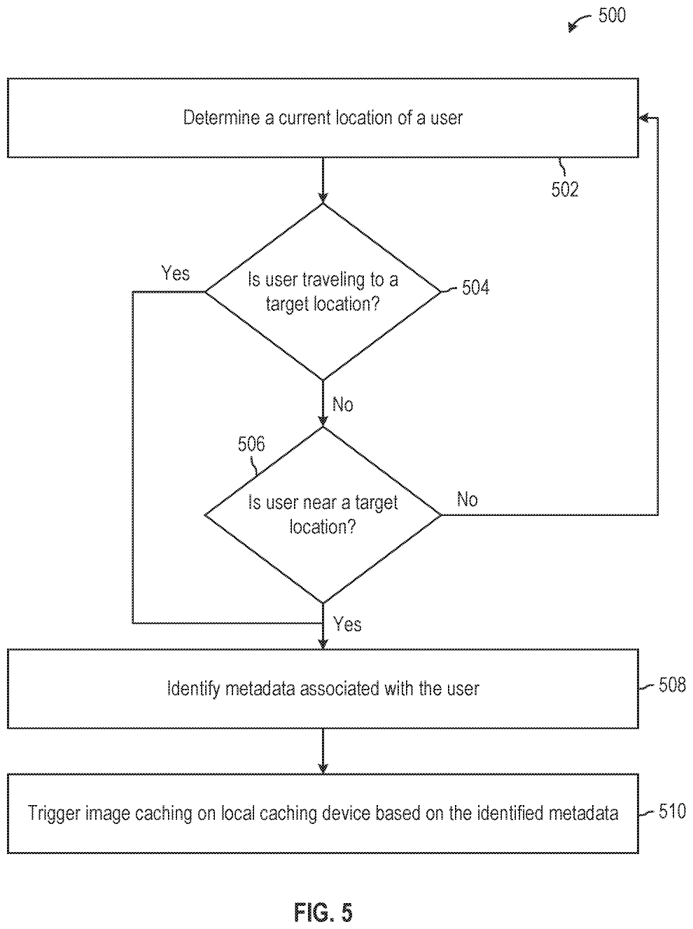

Referring now to FIG. 5, a process 500 for triggering image caching based on a user's location is shown, according to some embodiments. As mentioned above, many systems and methods cache image data based solely on static (i.e., predefined and/or unchanging) rules or policies, which often cache the data at inopportune or inefficient times. Further, some systems and methods do not cache image data prior to a user interacting with a viewing device, causing frustrating and time-consuming performance issues. In contrast, process 500 triggers image caching based on a user's actual location or a predicted future location to ensure that data is not cached too early or too late, thereby preserving valuable computation resources and increasing viewing performance. Process 500 may be especially advantageous in a healthcare setting, where users such as physicians may view large numbers of high-quality medical images on local image viewers. In some embodiments, one or more steps of process 500 may be implemented by PACS 106, as described above; however, it will be appreciated that certain steps may also be performed by image viewer 102, local caching device 202, and/or user device 302. It will also be appreciated that certain steps of process 500 may be optional and, in some embodiments, process 500 may be implemented using less than all of the steps.

At step 502, a current location of the user is determined. In some embodiments, the current location of the user is determined based on the location of a user device carried by the user. For example, the user may carry a smartphone, tablet, smartwatch, or other portable computing device that is capable of determine its location. In some such embodiments, the user device may be configured to determine a current location using GPS data, by triangulating a position using other types of radio signals (e.g., cellular), or by any other suitable method of determining a geographical location (e.g., using a local positioning system (LPS), etc.). Accordingly, the user's current location may be represented using geographical coordinates (i.e., longitude, latitude, and/or altitude) or by another suitable format. As mentioned above, for example, the user's location (i.e., location data) may also include a velocity of the user and/or a time at which the location data was determined.

In some embodiments, location data is subsequently transmitted a remote system (e.g., PACS 106). However, in other embodiments, location data is kept local to the user device (e.g., stored on the user device) to improve security and privacy for the user. In other words, in such embodiments, the user's location data is not transmitted to or stored on any remote system (e.g., PACS 106). Accordingly, in some embodiments, certain subsequent steps of process 500 (e.g., steps 504 and 506) may be implemented by user device 302 rather than PACS 106.

In some embodiments, location data is determined and/or transmitted to the remote system in real-time (i.e., on-demand). In other embodiments, location data is determined continuously or at a regular interval (e.g., every second, every minute, etc.). In some embodiments, rather than determining a user's location based on location data transmitted by a user device, the user may carry a card, badge, tag, or other device capable of wireless transmitting an identifier, which can then be detected by an external positioning system. For example, a physician may carry an identification badge that includes a radiofrequency identification (RFID) transceiver that can be detected by one or more external RFID readers.

In yet other embodiments, the user's location can be tracked using a building's internal security or access control system. For example, the location of the user may be determined or updated each time the user swipes an ID badge at an access point or enters a password on an electronic lock to enter a room. In yet other embodiments, the user's location may be predicted based on information from a remote system, such as a scheduling system or an electronic calendar. In a hospital, for example, a patient and/or staff schedule may be evaluated to determine when certain users are scheduled to work or to identify patients that a user is scheduled to meet. More specifically, a schedule of events, such as surgeries, appointments, meetings, etc., may be used to predict a user's location at a given time. For example, it may be determined that a physician is scheduled to be in a specific operating or examination room based on the hospital's scheduling system. Thus, it can be predicted that the physician will be in the identified operating or examination room at a specific time.

At step 504, the location data is user to determine whether the user is traveling (i.e., moving towards) an image viewing device (e.g., workstation), also referred to herein as a “target location.” In some embodiments, user location data may be collected at multiple time steps or over a period of time to determine a trajectory (e.g., heading and speed) of the user. For example, location data collected at first and second time steps may be compared to determine whether the user was moving and, if so, the trajectory of the user. Based on the user's trajectory, a future location of the user can be predicted. Predicted future locations for the user may then be compared to the locations of known image viewing devices to determine whether the user will be near a target location at a future time step.

In some embodiments, data indicating a sequence of locations of the user is collected over time. This data may include location data collected at one or more time steps over a period of time, along with a date, time, and/or speed of the user at each time step (i.e., at each location). In some such embodiments, sequence data is collected overtime and compared to known instances of the user reaching a target location or known target locations in order to identify sequences that lead the user to a target location. For example, location data may be collected over time to identify sequences or “paths” that the user has followed which lead to an image viewing device. These identified sequences may then be stored (e.g., in a database) for later comparison with live user location data. In some embodiments, for example, the location data collected at step 502 may be compared to a database of sequences that lead to target locations to determine whether the user is actively following a previously-defined sequence.

In some embodiments, identified sequences that lead to an image viewing device are used to train a neural network, such as a recurring neural network (RNN). In some such embodiments, the location data, dates, times, and/or speeds that are collected over time to form a sequence or “path” may be used as inputs to the RNN or another type of model. Sequences that are known to lead to a target location may then be used to train the model to output a prediction of whether a collected sequence will lead the user to an image viewing device. Thus, as location data is collected (e.g., at step 502), it may be fed into the model to predict the sequence or path the user is following, which can then be used to predict whether the user is heading towards a target location.

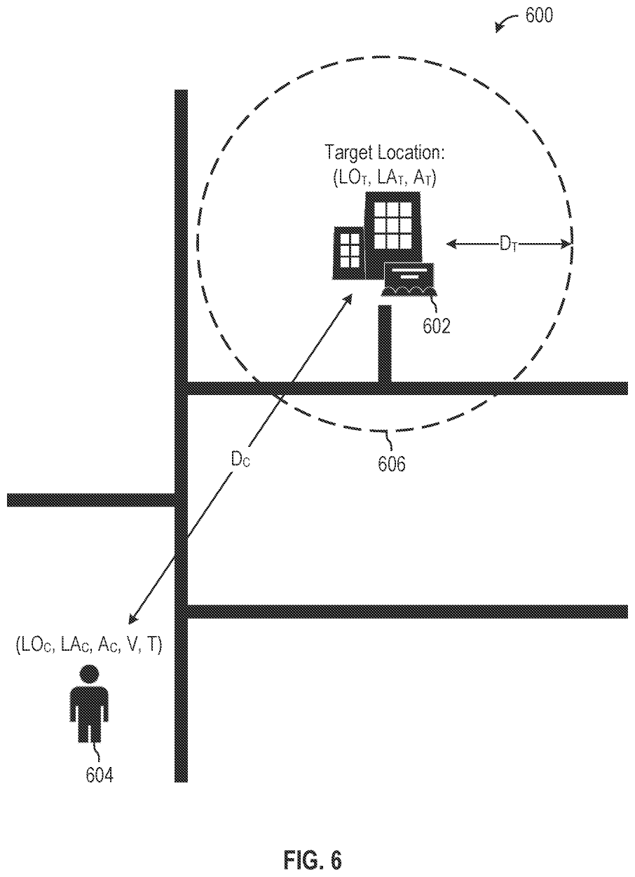

Turning briefly to FIG. 6, a diagram of illustrating the detection and analysis of a user's location using a RNN or other suitable machine learning algorithm is shown, according to some embodiments. Specifically, FIG. 6 includes a map 600 of an area that indicates a target location 602 (e.g., a hospital) and a location of a user 604 in relation to target location 602. Also shown surrounding target location 602 is a triggering distance identifier 606, which illustrates a distance from target location 602 at which the user's location can trigger image caching. As shown, triggering distance identifier 606 is defined by a trigger distance, D_{T}. Additionally, user 604 is shown to be separated from target location 602 by a current distance, D_{c}.

To determine the position of user 604 with respect to target location 602 and, more specifically, to determine whether user 604 is within the triggering distance, D_{T}, of target location 602 (e.g., to initiate the caching of image data), a current distance, D_{c}, between user 604 and target location 602 may be determined at regular intervals (e.g., every 10 msec, every minute, every hour, etc.). The current distance, D_{c}, may be defined as:

D_{C}=g(LO_{C},LA_{C},A_{C},LO_{T},LA_{T},A_{T})

where LO_{C}, LA_{C}, and A_{C} are the current latitude, longitude, and latitude of user 604, respectively, and LO_{T}, LA_{T}, and A_{T} are the latitude, longitude, and latitude of target location 602. Thus, determining a current distance, D_{C}, at regular time intervals may result in a sequence of distances (e.g., D_{C,1}, D_{C,2}, . . . D_{C,n}) between user 604 and target location 602, where each distance (e.g., D_{C,n}) is associated with a time step.

In some embodiments, said determination of current distance, D_{C}, at regular intervals may be triggered by a determination that the current distance, D_{C}, of user 604 to target location 602 is less than or equal to an initial distance, I_{D}. In some such embodiments, D_{C} is determined at a first time interval (e.g., every 10 minutes) when D_{C}>I_{D} and at a second time interval (e.g., every minute) when D_{C}≤I_{D}, where the second time interval is shorter than the first time interval. In this manner, only a small portion of the computing resources of user device 302 and/or PACS 106 are used prior to determining that user 604 is within the initial distance, I_{D}, of target location 602 by requiring less frequent distance calculations.

In some embodiments, the input to the machine learning algorithm (e.g., RNN) is a tuple defined as:

(LO_{C},LA_{C},A_{C},V,T)

where LO_{C}, LA_{C}, and A_{C} are the latitude, longitude, and latitude of user 604, respectively, V is the velocity of user 604, and T is the current time (i.e., the time at which LO_{C}, LA_{C}, and A_{C} were determined) for a single time step. In some such embodiments, V may be determined based on data from a secondary sensor (e.g., an accelerometer) and/or based on at least one of LO_{C}, LA_{C}, and A_{C} as determine at a previous time step.

In this manner, sequences of tuples (e.g., (LO_{C,1}, LA_{C,1}, A_{C,1}, V₁, T₁), (LO_{C,2}, LA_{C,2}, A_{C,2}, V₂, T₂), . . . (LO_{C,n}, LA_{C,n}, A_{C,n}, Vₙ, Tₙ)) for n time steps may be provided as inputs to the machine learning algorithm to determine a direction and/or speed that user 604 is moving. For example, the input for each recurrent layer of a RNN may be a single tuple associated with a particular time step. Accordingly, the output of the machine learning algorithm may be a predicted direction and speed of travel for user 604. Said prediction may then be compared to the known parameters for target location 602 (e.g., LO_{T}, LA_{T}, A_{T}) to determine whether user 604 is moving towards target location 602.

In some embodiments, once it has been determined that user 604 is moving towards target location 602, the current distance, D_{C}, of user 604 may be compared to the triggering distance, D_{T}, to determine when to trigger image caching. For example, once D_{C}≤D_{T}, image caching may be triggered. In some such embodiments, the predicted direction and/or speed of movement of user 604 may be utilized in conjunction with the condition D_{C}≤D_{T} to generate a binary output indicating whether user 604 is determined to be traveling to target location 602. For example, the predicted direction and/or speed of movement of user 604 and the output of condition D_{C}≤D_{T} may result in “1” or “TRUE” when it is determined that user 604 is determined to be traveling to target location 602.

In some embodiments, the machine learning algorithm described above for determining the user's trajectory and/or for detecting when the user has crossed a triggering threshold (e.g., D_{T}) may be trained using a set of known-good training data. In such embodiments, the training data may include a plurality of location sequences (e.g., sequences of tuples) that are known to have been followed by a user to reach a target location. In other words, the machine learning algorithm may be trained to predict the user's trajectory (i.e., direction of movement and speed) based on historical data. In some embodiments, the output of the machine learning algorithm may also be utilized to identify a particular caching location associated with target location 602. For example, the latitude, longitude, and latitude (e.g., LO_{T}, LA_{T}, and A_{T}) of a plurality of target locations may be stored in a database which can be accessed by the user's device (e.g., user device 302). Accordingly, the current latitude, longitude, and latitude (e.g., LO_{C}, LA_{C}, and A_{C}) of user 604 may be compared to the latitude, longitude, and latitude (e.g., LO_{T}, LA_{T}, and A_{T}) of each of the plurality of target locations to identify the target location that the user is traveling towards, or that the user is nearest to.

Referring again to FIG. 5, if the user is determined to be travelling to a target location (e.g., an image viewing device), then process 500 may continue to step 508, as described in greater detail below. If the user is not predicted to be travelling to a target location, or in instances when an insufficient amount of location data has been collected to generate an accurate prediction, the user's current location (e.g., at an individual time step) may be compared to location data for known target locations to determine if the user is near (e.g., within D_{T}) a target location. In some embodiments, location data for these target locations is stored in a look-up table on a database (e.g., in PACS 106). Accordingly, the user's location may be used to query the look-up table for a nearest image viewing device and/or the location data for the target locations may be referenced or downloaded by user device 302.

In some embodiments, the user may be considered to be “near” a target location if the user is within a threshold distance (e.g., D_{T}) from the target location. For example, an image viewing device may only be considered as “nearby” if it is within 500 feet of the user. In another example, all target locations within the same building as the user may be considered. If multiple image viewing devices are identified that are within the threshold distance, D_{T}, of the user, a closest (i.e., nearest) device may be identified as the target location. In some embodiments, if the user is not predicted to be near a target location within a predetermined amount of time (e.g., 90 seconds, 10 minutes, etc.) or if the user is determined not to be traveling (i.e., moving), process 500 may revert back to step 502 and/or may be terminated.

At step 508, metadata associated with the user is identified. In particular, an identifier for the user may be used to identify metadata (e.g., stored in a remote database) associated with that user. An identifier may be any set of alphanumeric characters or other symbols that identify the particular user, such as a name, an I_{D} number, an email address, a phone number, etc. However, transmitting personal identifying information (PII) may constitute a significant security risk. Thus, in some embodiments, only an I_{D} number associated with the user is identified and/or transmitted to the PACS. In some embodiments, a user's identifier is received at step 502 with location data. In other embodiments, the user's identifier is received before or after the location data. For example, a physician's I_{D} number or login credentials (e.g., to a hospital computer system) may be used to identify metadata associated with the physician.

Metadata may include information about the user themselves (e.g., name, email address, phone number, I_{D} number, etc.) as well as information relating to patients of the user (e.g., patient names, dates of birth, characteristics, appointments, medical history, etc.). Subsequently, the identified metadata may be used to identify image data associated with the user (e.g., physician) and the user's patients. For example, metadata and image data may be collocated in a database or may include pointers to corresponding metadata or image data. In this manner, image data and patient data associated with a particular physician may be quickly identified within a database.

At step 510, image caching on the identified local caching device is triggered. As described briefly above, once metadata and image data associated with the user is identified, and it is determined that the user is near or traveling to an image viewing device, image data caching may be triggered. In some embodiments, triggering image data caching causes a local caching device (e.g., an on-site server) to being downloading relevant image data and/or corresponding metadata. For example, the image caching device may be commanded to download all image data and metadata associated with the user (e.g., based on the user's identifier) or at least a subset of the user's associated image data and corresponding metadata.

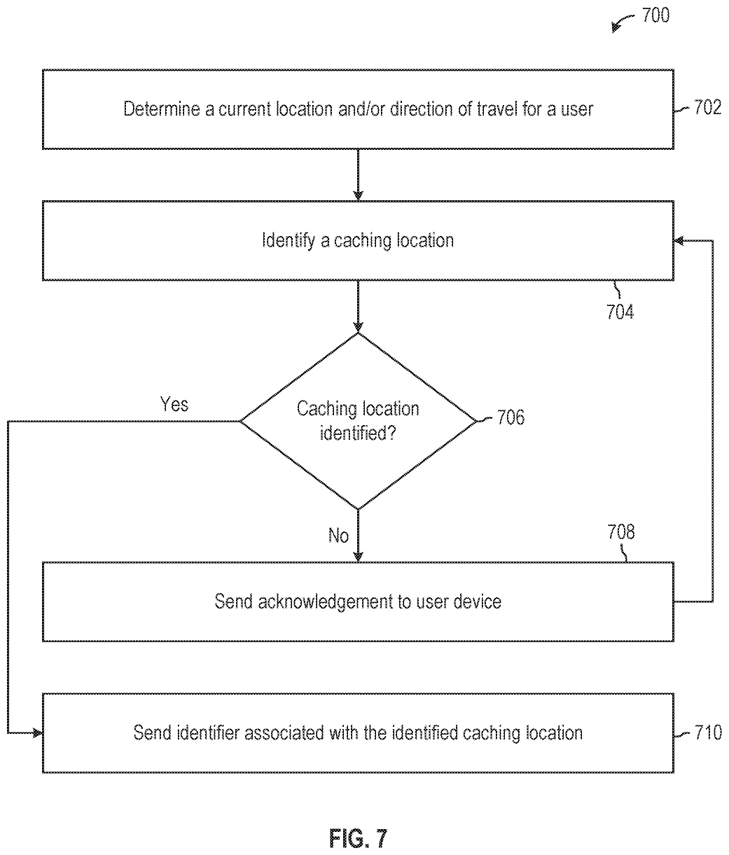

Referring now to FIG. 7, a process 700 for identifying a caching location based on a user's location is shown, according to some embodiments. In some cases, process 700 may be implemented in response to the triggering of image caching based on a user's location. For example, once it is determined that the user is at or near a local caching or image viewing device, process 700 may be initiated to identify a particular caching location. In some embodiments, one or more steps of process 700 may be implemented by PACS 106, as described above; however, it will be appreciated that certain steps may also be performed by image viewer 102, local caching device 202, and/or user device 302. It will also be appreciated that certain steps of process 700 may be optional and, in some embodiments, process 700 may be implemented using less than all of the steps.

At step 702, a current location and/or trajectory (i.e., direction of travel and speed) of a user is determined. In some embodiments, the current location of the user is determined based on the location of a user device carried by the user. For example, the user may carry a smartphone, tablet, smartwatch, or other portable computing device that can determine its location using GPS, by triangulating a position using other types of radio signals (e.g., cellular), or by any other suitable positioning method. In some embodiments, the user's device can execute a software application that can determine and/or track the user's location and speed. For the sake of brevity, it will be appreciated that step 702 may be similar to or the same as steps 502 and 504 of process 500, described above.

At step 704, a caching location is identified based on the user's current and/or predicted location. In some embodiments, as also discussed above, the user's location may be compared to location data for one or more known image viewing devices, which may be associated with corresponding caching devices (e.g., local caching devices). For example, a given image viewer may be communicably coupled to one or more caching devices via a network. In some embodiments, location data for local caching devices is stored in a database (e.g., in PACS 106). Accordingly, the user's location may be used to query the database for a nearest image viewing device or local caching device.

In some embodiments, a multinomial classification algorithm may be executed to identify a caching device. In some embodiments, a particular cache within a caching device may be identified. In some embodiments, each caching device and/or cache may be associated with a particular identifier, such as a web address or URL. Accordingly, the user's location may be compared (e.g., using a classification algorithm) to known caching devices and/or caches to identify a particular device or cache for caching image data. In some embodiments, a machine learning algorithm is used to identify a caching location by comparing the user's current location with the locations of one or more known image viewing devices or local caching devices, as described above with respect to FIG. 6.

If a caching location cannot be identified based on the user's location then, at step 708, an acknowledgement signal (“ACK”) may be transmitted to the user's device and process 700 may return to step 704. A caching location may not be identified for any number of reasons. For example, all of the caching devices for a given location (e.g., a particular hospital) may be in use or may lack adequate resources (e.g., available memory) to cache image data. In some embodiments, as described above with respect to FIG. 5, it may be determined that the user is not within a threshold distance of and/or is not traveling to an image viewing device or a local caching device. However, if a caching location can be identified then, at step 710, an identifier for the caching location may be transmitted to the user's device. The identifier, as mentioned above, may be any suitable identification of the particular caching device or cache to which image data should be downloaded. For example, the identifier may be a web address, which may be transmitted (i.e., sent) to the user's device.

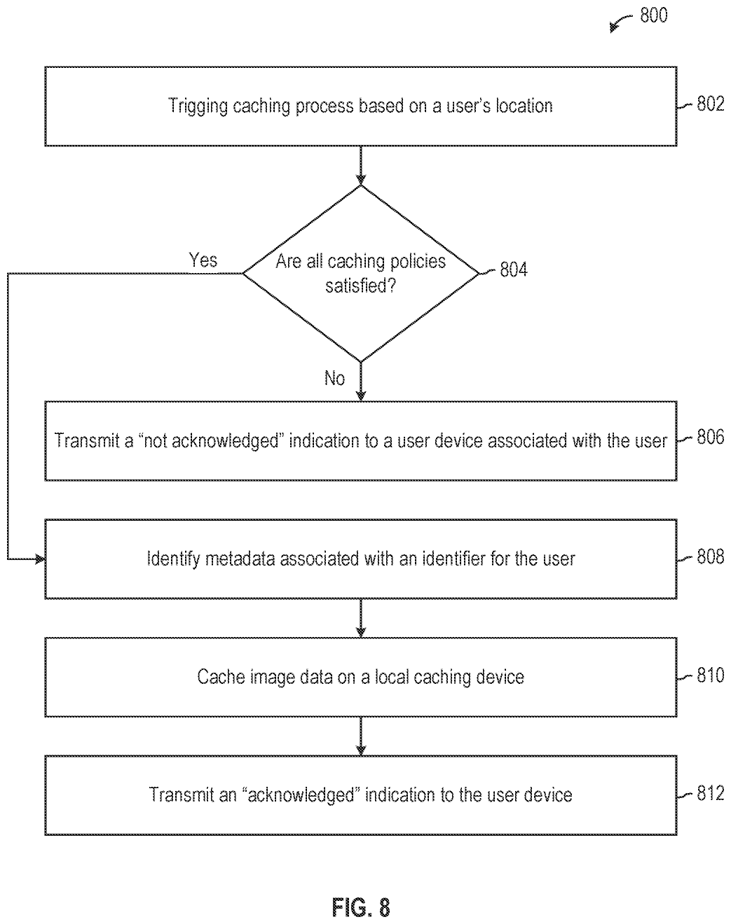

Referring now to FIG. 8, a process 800 for caching image data is shown, according to some embodiments. In particular, process 800 may be implemented in response to the triggering of image caching based on a user's location. Accordingly, image data may be cached on a local device for subsequent viewing (e.g., on an image viewing device). In general, only image data associated with the particular user is cached. In this regard, an identifier for the user may be used to identify image data and metadata associated with the user, as described above. In some embodiments, one or more steps of process 800 may be implemented by PACS 106, as described above; however, it will be appreciated that certain steps may also be performed by image viewer 102, local caching device 202, and/or user device 302. It will also be appreciated that certain steps of process 800 may be optional and, in some embodiments, process 800 may be implemented using less than all of the steps.

At step 802, image caching is triggered based on a location of a user. For the sake of brevity, it will be appreciated that the user's location and/or trajectory may be determined using the methods outlined above in steps 502 and 504 of process 500, or step 702 of process 700. If it is determined that the user is near (e.g., within a threshold distance of) or traveling to an image viewing device (e.g., a workstation in a hospital), image caching may be triggered. In some embodiments, the location data is used to identify a nearest available local caching device. Subsequently, an identifier for the caching device (e.g., a URL) may be transmitted to the user's device. The user's device may then use the identifier to send a “start caching” command signal, causing the local caching device to being caching image data.

At step 804, it is determined whether one or more static caching policies are satisfied. In particular, the one or more static caching policies may be evaluated prior to downloading image data to the local caching device. For example, the local caching device or a remote system (e.g., PACS 106) may check whether any static caching policies exist prior to downloading image data. Static caching policies may include rules that supersede the triggering of image caching based solely on the user's location. If the one or more static caching policies are not satisfied then, at step 806, a negative acknowledgement signal (“NACK”) may be transmitted to the user's device. Subsequently, in some embodiments, process 800 returns to step 802. In other embodiments, process 800 may be terminated or another process may be initiated to satisfy the caching policies.

If the one or more static caching policies are satisfied then, at step 808, metadata may be identified for the user. In particular, a list of studies may be identified from a database (e.g., maintained by PACS 106) based on an identifier for the user (e.g., a physician). As described above, studies are sets of information relating to a patient or procedure and corresponding sets of electronic medical images. For example, a physician's identification number or login credentials may be used to query a database of studies to identify studies associated with the particular physician. Once the studies associated with the physician are identified, the corresponding image data can be cached.

At step 810, image data is cached on a local caching device. In other words, one or more caching task may be performed (e.g., by PACS 106 and/or local caching device 202) to cache the image data on the local caching device. For example, the local caching device may query a database using the metadata identified at step 808 to identify image data associated with a particular user. Subsequently, the local caching device may begin to download the image data into a particular cache, or into a particular database. In some embodiments, image data may also be cached or downloaded to an image viewing device. Finally, at step 812, an acknowledgement signal (“ACK”) is transmitted to the user's device, indicating the successful caching of the image data.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A system for caching images for remote viewing, the system comprising:
a memory device having instructions stored thereon that, when executed by one or more processors, cause the one or more processors to perform operations comprising:
receiving first location data from a user device associated with a user;
determining whether the user is within a threshold distance of a remote image viewing device based on the first location data;
identifying a local caching device associated with the remote image viewing device responsive to a determination that the user is within the threshold distance of the remote image viewing device; and
transmitting image data associated with the user to the local caching device.

2. The system of claim 1, the operations further comprising:
determining an identifier for a particular cache of the local caching device; and
transmitting the identifier to the user device, wherein the image data is transmitted to the particular cache based on the identifier.

3. The system of claim 2, wherein the identifier is a web address or a link for the particular cache of the local caching device.

4. The system of claim 1, the operations further comprising:
receiving, from the user device, metadata associated with the user; and
identifying the image data associated with the user based on the metadata.

5. The system of claim 4, wherein identifying the image data further comprises:
querying an image database based on the metadata associated with the user; and
retrieving the image data from the image database.

6. The system of claim 1, wherein the first location data is received at a first time, the operations further comprising:
receiving second location data from the user device at a second time that is after the first time; and
determining whether the user is traveling to the remote image viewing device based on the first location data and the second location data, wherein the location caching device is further identified responsive to a determination that the user is traveling to the remote image viewing device.

7. The system of claim 1, the operations further comprising transmitting an acknowledgement to the user device responsive to identifying the local caching device.

8. The system of claim 1, wherein the local caching device is a computing device located at a healthcare facility.

9. The system of claim 1, wherein the image data is medical image data associated with one or more patients of the user.

10. A method of caching image data, the method comprising:
receiving, by a processor, location data from a mobile device associated with a user;
determining, by the processor, whether the user is within a threshold distance of a remote image viewing device;
identifying, by the processor, a caching service associated with the remote image viewing device responsive to a determination that the user is within the threshold distance of the remote image viewing device; and
transmitting, by the processor, the image data to the caching service.

11. The method of claim 9, further comprising:
determining, by the processor, an identifier for a particular cache of the caching service; and
transmitting, by the processor, the identifier to the user device, wherein the image data is transmitted to the particular cache based on the identifier.

12. The method of claim 10, wherein the identifier is a web address or a link for the particular cache.

13. The method of claim 9, further comprising:
receiving, by the processor, metadata associated with the user from the user device; and
identifying, by the processor, the image data associated with the user based on the metadata.

14. The method of claim 12, wherein identifying the image data further comprises:
querying, by the processor, an image database based on the metadata associated with the user; and
retrieving, by the processor, the image data from the image database.

15. The method of claim 9, wherein the first location data is received at a first time, the method further comprising:
receiving, by the processor, second location data from the user device at a second time that is after the first time; and
determining, by the processor, whether the user is traveling to the remote image viewing device based on the first location data and the second location data, wherein the caching service is further identified responsive to a determination that the user is traveling to the remote image viewing device.

16. The method of claim 9, further comprising transmitting an acknowledgement to the user device responsive to identifying the caching service.

17. The method of claim 9, wherein the caching service is hosted by a computing device located at a healthcare facility.

18. The method of claim 9, wherein the image data is medical image data associated with one or more patients of the user.

19. A non-transitory computer readable media having instructions stored thereon that, when executed by one or more processors, cause the one or more process to perform operations comprising:
continuously receiving location data from a user device associated with a user;
determining, based on the continuously received location data, whether the user is i) within a threshold distance of a remote image viewing device, or ii) traveling to the remote image viewing device;
identifying a local image caching device associated with the remote image viewing device responsive to a determination that the user is i) within the threshold distance of the remote image viewing device, or ii) traveling to the remote image viewing device;
identifying image data associated with the user; and
transmitting the image data to the local image caching device.

20. The computer readable media of claim 19, wherein the location data is received at a regular time interval.
